(19) United States
(12) Patent Application Publication
(10) Pub. No.: US 2009/0232896 A1
(43) Pub. Date: Sep. 17, 2009

(54) Use of Magnesium Stearate Dihydrate for Lubrication of Solid Pharmaceutical Compositions

(75) Inventors: Stephen H. Wu, Chesterfield, MO (US); Brian K. Cheng, Chesterfield, MO (US); Gary A. Nichols, Wildwood, MO (US); Jae H. Park, Olivette, MO (US)

Correspondence Address: Mallinckrodt Inc., 675 McDonnell Boulevard, HAZELWOOD, MO, 63042, US

(73) Assignee: Mallinckrodt Inc., Hazelwood, MO (US)

(21) Appl. No.: 12/369,013

(22) Filed: Feb. 11, 2009

Related U.S. Application Data
(60) Provisional application No. 61/027,580, filed on Mar. 11, 2008.
(60) Provisional application No. 61/042,001, filed on Apr. 3, 2008.
(60) Provisional application No. 61/055,157, filed on May 22, 2008.

(51) Int. Cl. A61K 9/14; A61K 47/14; A61K 47/12

(52) U.S. Cl. 424/490; 514/785; 514/784

(57) ABSTRACT

The invention provides lubricant compositions comprising magnesium stearate dihydrate. The lubricant compositions may be used to lubricate a variety of bioactive formulations including pharmaceutical compositions.

### FIELD OF THE INVENTION

The invention generally relates to lubricant compositions and methods for lubricating solid materials. In particular, the invention provides magnesium stearate dihydrate compositions that may be used to lubricate pharmaceutical compositions.

### BACKGROUND OF THE INVENTION

Lubricants are used in powder blending and tablet production for their anti-adherent activity (i.e., prevent sticking to punch faces and die walls), glidant activity (i.e., improve the flowability of the powder or granules), and lubricant activity (i.e., reduce friction at the tablet-die wall interface during compaction and ejection during the tableting process). Magnesium stearate (MgSt) is widely used as a lubricant in the manufacture of medical tablets and capsules. MgSt has advantages over other lubricants because of its high melting temperature, high lubricity at a low concentration, large covering potential, general acceptance as safe, nontoxicity, and its excellent stability profile.

Magnesium stearate is commercially available mainly in the monohydrate form (MgSt-M) or as a mixture of the monohydrate along with trace amounts of other crystalline forms, such as the dihydrate (MgSt-D) and trihydrate, and amorphous forms. The composition of MgSt preparations not only varies from manufacturer to manufacturer, but also from lot-to-lot. Thus, variations in the composition of MgSt preparations and the different crystalline states of the various hydrate forms could affect the uniformity of the ingredients blended together, as well as the compressibility and quality of the resulting tablets. Because of the variations in the compositions of MgSt preparations, there is a need for pure forms of MgSt. Furthermore, there is a need for methods of using MgSt-D as a lubricant in the dry blending and tableting of pharmaceutically active ingredient(s) with diluents and other exipients.

### SUMMARY OF THE INVENTION

One aspect of the invention provides a method for lubrication of a pharmaceutical composition. Typically, the pharmaceutical composition comprises at least one pharmaceutically active ingredient and at least one excipient. The method comprises combining the pharmaceutical composition with a lubricant composition comprising at least 40% by weight of magnesium stearate dihydrate.

Another aspect of the invention encompasses a pharmaceutical composition. The pharmaceutical composition comprises at least one pharmaceutically active ingredient and a lubricant composition comprising at least 40% by weight of magnesium stearate dihydrate.

Yet another aspect of the invention provides a composition comprising a lubricant composition comprising at least 40% by weight of magnesium stearate dihydrate and at least one excipient.

Other features and iterations of the invention are described in more detail below.

### DESCRIPTION OF THE FIGURES

FIG. 1 presents the powder X-ray diffraction patterns of the monohydrate and dihydrate forms of magnesium stearate (MgSt).

FIG. 2 presents scanning electron micrographs of MgSt-monohydrate (A) and MgSt-dihydrate (B).

FIG. 3 presents the differential scanning calorimetry profiles of the monohydrate and dihydrate forms of MgSt.

FIG. 4 shows the thermogravimetric analysis for the monohydrate and dihydrate forms of MgSt.

FIG. 5 presents the near-infrared spectra of the monohydrate and dihydrate forms of MgSt.

FIG. 6 presents an in-line effusivity plot of (four) baseline runs with neat microcrystalline cellulose.

FIG. 7 presents an effusivity plot of Batch 7 (acetaminophen-microcrystalline cellulose-dibasic calcium phosphate (APAP-MCC-DCP) lubricated with 1% MgSt-M).

FIG. 8 presents an effusivity plot of Batch 11 (APAP-MCC-DCP lubricated with 1% MgSt-D).

FIG. 9 presents an effusivity plot of Batch 12 (APAP-MCC-lactose monohydrate (APAP-MCC-LAC) lubricated with 1% MgSt-M).

FIG. 10 presents an effusivity plot of Batch 14 (APAP-MCC-LAC lubricated with 1% MgSt-D).

FIG. 11 presents a scatterplot analysis of the average compression coefficient 50 mm/s as a function of blend time for the monohydrate and dihydrate forms of MgSt.

FIG. 12 presents a scatterplot analysis of the standard deviation of compression as a function of blend time for the monohydrate and dihydrate forms of MgSt.

FIG. 13 is a main effects plot for effusivity as a function of MgSt type and percent of MgSt.

FIG. 14 is a main effects plot for total compression forces as a function of MgSt type and percent of MgSt.

FIG. 15 is a main effects plot for ejection force as a function of MgSt type and percent of MgSt.

FIG. 16 plots the % compressibility of blends comprising starch as a diluent. MG1 refers to MgSt-M and MG2 refers to MgSt-D.

FIG. 17 presents dissolution profiles for APAP-MCC-DCP blends with 0.3% MgSt.

FIG. 18 presents dissolution profiles for APAP-MCC-DCP blends with 1.0% MgSt.

FIG. 19 presents dissolution profiles for APAP-MCC-LAC blends with 1.0% MgSt.

### DETAILED DESCRIPTION OF THE INVENTION

The present invention provides lubricant compositions, and methods of using the lubricant compositions to lubricate solid materials. In particular, the lubricant compositions may be used to lubricate excipients or blends of pharmaceutically active ingredients and excipients. Typically, the lubricant composition comprises at least 40% by weight MgSt-D. In particular, it has been discovered, as illustrated in the examples, that MgSt-D provides certain advantages as a lubricant when compared to MgSt-M. For example, the use of MgSt-D for pharmaceutical applications generally achieves comparable blend uniformity of the pharmaceutically active ingredients and excipients in a shorter blending time compared to MgSt-M. Moreover, the blend uniformity is typically less sensitive to blending time, and the mixture generally exhibits improved lubricating efficiency in subsequent tableting processes when MgSt-D is used as a lubricant compared to the use of MgSt-M. Furthermore, the use of MgSt-D as a lubricant minimizes capping, sticking, and lamination problems during the tablet making process. In view of the desirable properties of MgSt-D, it may be beneficially used as a lubricant in pharmaceutical compositions.

### I. Lubricant Compositions

The lubricant compositions comprise MgSt-D. The amount of MgSt-D comprising the lubricant composition can and will vary depending upon the application. Typically, the lubricant composition will include at least 40% by weight of MgSt-D. In other embodiments, the lubricant composition will include at least 45%, 50%, 55%, 60%, 65%, 70%, 75%, 80%, 85%, 90%, 95%, 97% or greater than 99% by weight of MgSt-D. In an exemplary embodiment, the lubricant composition will comprise greater than 90% by weight of MgSt-D. In each of the foregoing embodiments, the lubricant composition typically will have less than about 5% by weight of MgSt-M. More typically, the lubricant composition will have less than about 4.5%, 4.0%, 3.5%, 3.0%, 2.5%, 2.0%, 1.5%, 1.0%, or less than about 0.5% by weight of MgSt-M. In an exemplary embodiment, the amount of MgSt-M in the lubricant composition is less than about 1.0% by weight.

Generally speaking, MgSt-D is composed of a mixture of stearic acid, palmitic acid, and water. The weight ratio of stearic acid, palmitic acid, and water can and will vary depending upon the manner in which the MgSt-D is made. In one embodiment, the weight ratio of stearic acid to palmitic acid may range from about 2:1 to 1:2. In an exemplary embodiment, the weight ratio of stearic acid to palmitic acid is about 2:1. MgSt-D having a weight ratio of stearic acid to palmitic acid of 2:1 may be manufactured according to the following two-step general scheme:

C₁₇H₃₅COOH+NaOH→C₁₇H₃₅COONa+H₂O; and

2C₁₇H₃₅COONa+MgSO₄.7H₂O→Mg(C₁₇H₃₅COO)₂.2H₂O+Na₂SO₄+6H₂O

Highly pure MgSt-D that is a crystalline form of matter and more particularly, is a stable polymorph may be manufactured using the reaction scheme detailed above in combination with the reaction conditions reported in U.S. Application Publication Nos. 2006/0281937 and 2006/0247456, both of which are incorporated herein by reference in their entirety.

As will be appreciated by a skilled artisan, the particle size of the MgSt-D can and will vary depending upon the solid material to be lubricated. The MgSt-D will, however, at least be generally the same particle size or smaller than the particle size of the solid material. In certain embodiments, the average diameter of the MgSt-D may range from about 1 to about 500 microns. In other embodiments, the average diameter of the MgSt-D may range from about 5 to about 250 microns. In another embodiment, the average diameter of the MgSt-D may range from about 5 to about 100 microns. Alternatively, the average diameter of MgSt-D may range from about 10 to about 50 microns. In other embodiments, the average diameter of the MgSt-D will be less than about 30 microns, less than about 25 microns, less than about 20 microns, less than about 15 microns, or less than about 10 microns.

In an exemplary embodiment, the MgSt-D particles may be less than about 30 microns in diameter and may have a D₅₀ (i.e., 50^{th} percentile of the particle size distribution) of about 11 microns to about 16 microns, a D₉₀ (i.e., 90^{th} percentile of the particle size distribution) of about 22 microns to about 28 microns, an surface area of about 4.0 m²/g to about 7.5 m²/g depending upon particle size. In another exemplary embodiment, the MgSt-D particles may be micronized, i.e., they are reduced to less than 10 microns in diameter by conventional milling processes. These micronized particles have a D₅₀ of about 5 microns, a D₉₀ of less than about 10 microns, and a surface area of about 10 m²/g to about 20 m²/g depending upon the particle size.

Typically, the lubricant compositions may be suitably employed to lubricate a wide variety of solid materials irrespective of their form or size. For example, the lubricant composition may be used to lubricate a solid surface not having a reduced particle size such as a glass surface, metal surface, clay surface, ceramic surface, or a plastic surface. Alternatively, the lubricant compositions may be employed to lubricate solid materials having a reduced particle size. Non-limiting examples of solid materials having reduced particle sizes include powders, beads, granules, crystals, and encapsulated materials (e.g., lyophilized liposomes, encapsulated liquids, encapsulated semisolids, or encapsulated solids).

### II. Lubrication of Solid Pharmaceutical Compositions

The lubricant compositions may be utilized to lubricate ingredients that form a pharmaceutical composition. The pharmaceutical composition will typically include at least one pharmaceutically active ingredient, at least one excipient, and the lubricant composition of the present invention.

The pharmaceutical composition will generally comprise from about 0.01% to about 10% by weight of the lubricant composition comprising MgSt-D. In another embodiment, the pharmaceutical composition will comprise from about 0.1% to about 5% by weight of the lubricant composition comprising MgSt-D. In a further embodiment, the pharmaceutical composition will comprise from about 0.5% to about 2% by weight of the lubricant composition comprising MgSt-D.

As will be appreciated by a skilled artisan, the amount of pharmaceutically active agent and other excipients (i.e., in addition to the lubricant composition) forming the pharmaceutical composition can and will vary without departing from the scope of the invention. The weight fraction of the pharmaceutically active ingredient, excipient or combination of excipients in the pharmaceutical composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less, or about 0.1% or less of the total weight of the pharmaceutical composition. Suitable pharmaceutically active agents and excipients are described below.

a. Pharmaceutically Active Ingredients

The pharmaceutically active ingredient may include several bioactive agents. Non-limiting bioactive agents include drugs, minerals, vitamins, herbs, and other ingredients generally known in the art that are utilized in nutraceuticals or nutritional products.

In one embodiment, the bioactive agent is a drug. Suitable drugs, include, without limitation, an opioid analgesic agent (e.g., as morphine, hydromorphone, oxymorphone, levophanol, methadone, meperidine, fentanyl, codeine, hydrocodone, oxycodone, propoxyphene, buprenorphine, butorphanol, pentazocine and nalbuphine); a non-opioid analgesic agent (e.g., acetylsalicylic acid, acetaminophen, paracetamol, ibuprofen, ketoprofen, indomethacin, diflunisol, naproxen, ketorolac, dichlophenac, tolmetin, sulindac, phenacetin, piroxicam, and mefamanic acid); an anti-inflammatory agent (e.g., glucocorticoids such as alclometasone, fluocinonide, methylprednisolone, triamcinolone and dexamethasone; and non-steroidal antiinflammatory drugs such as celecoxib, deracoxib, ketoprofen, lumiracoxib, meloxicam, parecoxib, rofecoxib, and valdecoxib); an antitussive agent (e.g., dextromethorphan, codeine, hydrocodone, caramiphen, carbetapentane, and dextromethorphan); an antipyretic agent (e.g., acetylsalicylic acid and acetaminophen); an antibiotic agent (e.g., aminoglycosides such as, amikacin, gentamicin, kanamycin, neomycin, netilmicin, streptomycin, and tobramycin; carbecephem such as loracarbef; carbapenems such as certapenem, imipenem, and meropenem; cephalosporins such as cefadroxil cefazolin, cephalexin, cefaclor, cefamandole, cephalexin, cefoxitin, cefprozil, cefuroxime, cefixime, cefdinir, cefditoren, cefoperazone, cefotaxime, cefpodoxime, ceftazidime, ceftibuten, ceftizoxime, and ceftriaxone; macrolides such as azithromycin, clarithromycin, dirithromycin, erythromycin, and troleandomycin; monobactam; penicillins such as amoxicillin, ampicillin, carbenicillin, cloxacillin, dicloxacillin, nafcillin, oxacillin, penicillin G, penicillin V, piperacillin, and ticarcillin; polypeptides such as bacitracin, colistin, and polymyxin B; quinolones such as ciprofloxacin, enoxacin, gatifloxacin, levofloxacin, lomefloxacin, moxifloxacin, norfloxacin, ofloxacin, and trovafloxacin; sulfonamides such as mafenide, sulfacetamide, sulfamethizole, sulfasalazine, sulfisoxazole, and trimethoprim-sulfamethoxazole; and tetracyclines such as demeclocycline, doxycycline, minocycline, and oxytetracycline); an antimicrobial agent (e.g., ketoconazole, amoxicillin, cephalexin, miconazole, econazole, acyclovir, and nelfinavir); a steroidal agent (e.g., estradiol, testosterone, cortisol, aldosterone, prednisone, and cortisone); an amphetamine stimulant agent (e.g., amphetamine); a non-amphetamine stimulant agent (e.g., methylphenidate, nicotine, and caffeine); a laxative agent (e.g., bisacodyl, casanthranol, senna, and castor oil); an anorexic agent (e.g., fenfluramine, dexfenfluramine, mazindol, phentermine, and aminorex); an antihistaminic agent (e.g., phencarol, cetirizine, cinnarizine, ethamidindole, azatadine, brompheniramine, hydroxyzine, and chlorpheniramine); an antiasthmatic agent (e.g., zileuton, montelukast, omalizumab, fluticasone, and zafirlukast); an antidiuretic agent (e.g., desmopressin, vasopressin, and lypressin); an antiflatulant agent (e.g., simethicone); an antimigraine agent (e.g., naratriptan, frovatriptan, eletriptan, dihydroergotamine, zolmitriptan, almotriptan, and sumatriptan); an antispasmodic agent (e.g., dicyclomine, hyoscyamine, and peppermint oil); an antidiabetic agent (e.g., methformin, acarbose, miglitol, pioglitazone, rosiglitazone, troglitazone, nateglinide, repaglinide, mitiglinide, saxagliptin, sitagliptine, vildagliptin, acetohexamide, chlorpropamide, gliciazide, glimepiride, glipizide, glyburide, tolazamide, and tolbutamide); an antacid (e.g., aluminium hydroxide, magnesium hydroxide, calcium carbonate, sodium bicarbonate, and bismuth subsalicylate); a respiratory agent (e.g., albuterol, ephedrine, metaproterenol, and terbutaline); a sympathomimetic agent (e.g., pseudoephedrine, phenylephrine, phenylpropanolamine, epinephrine, norepinephrine, dopamine, and ephedrine); an H₂ blocking agent (e.g., cimetidine, famotidine, nizatidine, and ranitidine); an antihyperlipidemic agent (e.g., clofibrate, cholestyramine, colestipol, fluvastatin, atorvastatin, genfibrozil, lovastatin, niacin, pravastatin, fenofibrate, colesevelam, and simvastatin ); an antihypercholesterol agent (e.g., lovastatin, simvastatin, pravastatin, fluvastatin, atorvastatin, cholestyramine, colestipol, colesevelam, nicotinic acid, gemfibrozil, and ezetimibe); a cardiotonic agent (e.g., digitalis, ubidecarenone, and dopamine); a vasodilating agent (e.g., nitroglycerin, captopril, dihydralazine, diltiazem, and isosorbide dinitrate); a vasoconstricting agent (e.g., dihydroergotoxine and dihydroergotamine); a sedative agent (e.g., amobarbital, pentobarbital, secobarbital, clomethiazole, diphenhydramine hydrochloride, and alprazolam); a hypnotic agent (e.g., zaleplon, zolpidem, eszopiclone, zopiclone, chloral hydrate, and clomethiazole); an anticonvulsant agent (e.g., lamitrogene, oxycarbamezine, phenytoin, mephenytoin, ethosuximide, methsuccimide, carbamazepine, valproic acid, gabapentin, topiramate, felbamate, and phenobarbital); a muscle relaxing agent (e.g., baclofen, carisoprodol, chlorzoxazone, cyclobenzaprine, dantrolene sodium, metaxalone, orphenadrine, pancuronium bromide, and tizanidine); an antipsychotic agent (e.g., phenothiazine, chlorpromazine, fluphenazine, perphenazine, prochlorperazine, thioridazine, trifluoperazine, haloperidol, droperidol, pimozide, clozapine, olanzapine, risperidone, quetiapine, ziprasidone, melperone, and paliperidone); an anfianxiolitic agent (e.g., lorazepam, alprazolam, clonazepam, diazepam, buspirone, meprobamate, and flunitrazepam); an antihyperactive agent (e.g., methylphenidate, amphetamine, and dextroamphetamine); an antihypertensive agent (e.g., alpha-methyldopa, chlortalidone, reserpine, syrosingopine, rescinnamine, prazosin, phentolamine, felodipine, propanolol, pindolol, labetalol, clonidine, captopril, enalapril, and lisonopril); an anti-neoplasia agent (e.g., taxol, actinomycin, bleomycin A₂, mitomycin C, daunorubicin, doxorubicin, epirubicin, idarubicin, and mitoxantrone); a soporific agent (e.g., zolpidem tartrate, eszopiclone, ramelteon, and zaleplon); a tranquilizer (e.g., alprazolam, clonazepam, diazepam, flunitrazepam, lorazepam, triazolam, chlorpromazine, fluphenazine, haloperidol, loxapine succinate, perphenazine, prochlorperazine, thiothixene, and trifluoperazine); a decongestant (e.g., ephedrine, phenylephrine, naphazoline, and tetrahydrozoline); a beta blocker (e.g., levobunolol, pindolol, timolol maleate, bisoprolol, carvedilol, and butoxamine); an alpha blocker (e.g., doxazosin, prazosin, phenoxybenzamine, phentolamine, tamsulosin, alfuzosin, and terazosin); a non-steroidal hormone (e.g., corticotropin, vasopressin, oxytocin, insulin, oxendolone, thyroid hormone, and adrenal hormone); a herbal agent (e.g., glycyrrhiza, aloe, garlic, nigella sativa, rauwolfia, St John's wort, and valerian); an enzyme (e.g., lipase, protease, amylase, lactase, lysozyme, and urokinase); a humoral agent (e.g., prostaglandins, natural and synthetic, for example, PGE₁, PGE₂alpha, PGE₂alpha, and the PGE₁ analog misoprostol); a psychic energizer (e.g., 3-(2-aminopropy)indole and 3-(2-aminobutyl)indole); and an anti-nausea agent (e.g., dolasetron, granisetron, ondansetron, tropisetron, meclizine, and cyclizine).

In a preferred embodiment, the drug may be an opioid analgesic agent (e.g., codeine, hydrocodone, hydromorphone, methadone, morphine, oxycodone, tramadol, and the like), a non-opioid analgesic agent (e.g., acetaminophen, aspirin, benzonatate, butalbital, meloxicam, and so forth), or combinations thereof. In another preferred embodiment, the drug may be an antidepressant (e.g., a tricyclic antidepressant such as imipramine, methylphenidate, or nortriptyline; or a selective serotonin uptake inhibitor such as fluoxetine or paroxetine).

In another embodiment, the bioactive agent is a mineral or a mineral source. Non-limiting examples of minerals include, without limitation, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof. Suitable forms of zinc, include, zinc chelates (complexes of zinc and amino acids, dipeptides, or polypeptides), zinc acetate, zinc aspartate, zinc citrate, zinc glucoheptonate, zinc gluconate, zinc glycerate, zinc picolinate, zinc monomethionine and zinc sulfate. Examples of suitable forms of copper include copper chelates, cupric oxide, copper gluconate, copper sulfate, and copper amino acid chelates. Suitable forms of calcium include calcium alpha-ketoglutarate, calcium acetate, calcium alginate, calcium ascorbate, calcium aspartate, calcium caprylate, calcium carbonate, calcium chelates, calcium chloride, calcium citrate, calcium citrate malate, calcium formate, calcium glubionate, calcium glucoheptonate, calcium gluconate, calcium glutarate, calcium glycerophosphate, calcium lactate, calcium lysinate, calcium malate, calcium orotate, calcium oxalate, calcium oxide, calcium pantothenate, calcium phosphate, calcium pyrophosphate, calcium succinate, calcium sulfate, calcium undecylenate, coral calcium, dicalcium citrate, dicalcium malate, dihydroxycalcium malate, dicalcium phosphate, and tricalcium phosphate. A variety of suitable forms of iron may be included in the pharmaceutical composition of the invention. In one embodiment, the iron may be in the form of chelates, such as FERROCHEL™ (Albion Intemational, Inc., Clearfield, Utah) a commercially available bis-glycine chelate of iron, and SUMALATE™ (Albion International, Inc., Clearfield, Utah) a commercially available ferrous asparto glycinate. Other suitable forms of iron for purposes of the present invention include for example but are not limited to soluble iron salts, slightly soluble iron salts, insoluble iron salts, chelated iron, iron complexes, non-reactive iron such as carbonyl iron and reduced iron, and combinations thereof.

In a further embodiment, the bioactive agent is a vitamin. Suitable vitamins for use in the pharmaceutical compositions include vitamin C, vitamin A, vitamin E, vitamin B12, vitamin K, riboflavin, niacin, vitamin D, vitamin B6, folic acid, pyridoxine, thiamine, pantothenic acid, and biotin. The form of the vitamin may include salts of the vitamin, derivatives of the vitamin, compounds having the same or similar activity of a vitamin, and metabolites of a vitamin.

In still another embodiment, the bioactive agent may be an herb or a botanical or herbal extract. Examples of suitable herbs for use in nutraceutical compositions include alfalfa, aloe vera, bdellium gum, bilberry, black cohash, black currant oil, cayenne, chamomile, chickweed, dandelion, Echinacea, elderberry flowers, fennel, flax seed oil, garlic, gentian root, ginger, ginkgo biloba, ginseng, hawthorne berries, hops, ho shou wu, hyssop, juniper berry, kelp, lobelia, mandrake, myrrh, passionflower, peppermint, queen of the meadow, safflower, sarsaparilla, saw palmetto, and suma. Other suitable botanically derived agents include anthocyanins, beta-carotene, clove extract, epigallocatechin gallate, flavones, flavonoids, alpha-linolenic acid, lutein, lycopene, omega-3 fatty acids, pimento extract, resveratrol, rice bran extract, rosemary extract, sage extract, vitamin Q10, and so forth.

b. Excipients

Non-limiting examples of suitable excipients include an agent selected from the group consisting of non-effervescent disintegrants, a coloring agent, a flavor-modifying agent, an oral dispersing agent, a stabilizer, a preservative, a diluent, a compaction agent, a lubricant, a filler, a binder, and an effervescent disintegration agent. The amount and types of excipients utilized to form pharmaceutical compositions may be selected according to known principles of pharmaceutical science.

In an exemplary embodiment, the exicipient will include at least one diluent. The diluent may be compressible (i.e., plastically deformable) or abrasively brittle. Examples of suitable compressible diluents/fillers include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lacitol, mannitol, malitol, sorbitol, xylitol, maltodextrin, and trehalose. Examples of suitable abrasively brittle diluents/fillers include dibasic calcium phosphate (anhydrous or dihydrate), calcium phosphate tribasic, calcium carbonate, and magnesium carbonate.

In one embodiment, the excipient may include a binder. Suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

The excipient may comprise a non-effervescent disintegrant. Suitable examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth.

In another embodiment, the excipient may include an effervescent disintegrant. By way of non-limiting example, suitable effervescent disintegrants include sodium bicarbonate in combination with citric acid and sodium bicarbonate in combination with tartaric acid.

The excipient may comprise a preservative. Suitable examples of preservatives include antioxidants, such as a-tocopherol or ascorbate, and antimicrobials, such as parabens, chlorobutanol or phenol.

The excipient may include flavors. Flavors may be chosen from synthetic flavor oils and flavoring aromatics and/or natural oils, extracts from plants, leaves, flowers, fruits, and combinations thereof. By way of example, these may include cinnamon oils, oil of wintergreen, peppermint oils, clover oil, hay oil, anise oil, eucalyptus, vanilla, citrus oil, such as lemon oil, orange oil, grape and grapefruit oil, fruit essences including apple, peach, pear, strawberry, raspberry, cherry, plum, pineapple, and apricot.

In another embodiment, the excipient may include a sweetener. By way of non-limiting example, the sweetener may be selected from glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; sugar alcohols such as sorbitol, mannitol, sylitol, and the like. Also contemplated are hydrogenated starch hydrolysates and the synthetic sweetener 3,6-dihydro-6-methyl-1,2,3-oxathiazin4-one-2,2-dioxide, particularly the potassium salt (acesulfame-K), and sodium and calcium salts thereof.

The excipient may be a dispersion enhancer. Suitable dispersants may include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

Depending upon the embodiment, it may be desirable to provide a coloring agent. Suitable color additives include food, drug and cosmetic colors (FD&C), drug and cosmetic colors (D&C), or external drug and cosmetic colors (Ext. D&C). These colors or dyes, along with their corresponding lakes, and certain natural and derived colorants may be suitable for use in the present invention depending on the embodiment.

c. Solid Dosage Forms

The pharmaceutical composition is generally formulated into a solid dosage form. Suitable dosage forms include a tablet, including a suspension tablet, a chewable tablet, an effervescent tablet or caplet; a pill; a powder such as a sterile packaged powder, a dispensable powder, and an effervescent powder; a capsule including both soft or hard gelatin capsules such as HPMC capsules; a lozenge; a sachet; a sprinkle; a reconstitutable powder or shake; a troche; pellets; granules; liquids; suspensions; emulsions; or semisolids and gels. Alternatively, the pharmaceutical compositions may be incorporated into a food product or powder for mixing with a liquid, or administered orally after only mixing with a non-foodstuff liquid.

The amount and types of pharmaceutically active ingredients (i.e., drug or nutrient), and other excipients useful in each of these dosage forms are described throughout the specification and examples. It should be recognized that where a combination of ingredients and/or excipient, including specific amounts of these components, is described with one dosage form that the same combination could be used for any other suitable dosage form. Moreover, it should be understood that one of skill in the art would, with the teachings found within this application, be able to make any of the dosage forms listed above by combining the amounts and types of ingredients administered as a combination in a single dosage form or a separate dosage forms and administered together as described in the different sections of the specification.

The particle size of the ingredients forming the pharmaceutical composition may be an important factor that can effect bioavailability, blend uniformity, segregation, and flow properties. In general, smaller particle sizes of a pharmaceutically active ingredient increase the bioabsorption rate of the drug or nutrient by increasing the surface area. The particle size of the drug or nutrient and excipients can also affect the suspension properties of the pharmaceutical formulation. For example, smaller particles are less likely to settle and therefore form better suspensions. In various embodiments, the average particle size of the dry powder of the various ingredients (which can be administered directly, as a powder for suspension, or used in a solid dosage form) is less than about 500 microns in diameter, or less than about 450 microns in diameter, or less than about 400 microns in diameter, or less than about 350 microns in diameter, or less than about 300 microns in diameter, or less than about 250 microns in diameter, or less than about 200 microns in diameter, or less than about 150 microns in diameter, or less than about 100 microns in diameter, or less than about 75 microns in diameter, or less than about 50 microns in diameter, or less than about 25 microns in diameter, or less than about 15 microns in diameter. In some applications the use of particles less than 15 microns in diameter may be advantageous. In these cases colloidal or nanosized particles in the particle size range of 15 microns down to 10 nanometers may be advantageously employed.

The pharmaceutical compositions of the present invention can be manufactured by conventional pharmacological techniques. Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., prilling, spray drying, pan coating, melt granulation, granulation, wurster coating, tangential coating, top spraying, extruding, coacervation and the like.

### III. Taste Masking of Pharmaceutical Active Ingredients

The MgSt-D compositions of the invention also may be used to mask an objectionable taste of a pharmaceutical active ingredient in a pharmaceutical composition. That is, coating or covering a particle of a pharmaceutical active ingredient with a material comprising MgSt-D effectively masks the taste of the active ingredient. The MgSt-D comprising composition of the invention, therefore, may serve a dual function of acting as a lubricating agent for the ingredients comprising a pharmaceutical composition and a taste masking agent for a pharmaceutical active ingredient.

The pharmaceutical active ingredient may have a bitter taste, an astringent taste, a metallic taste, a medicinal taste, or another “unpleasant” taste. As detailed below, the objectionable taste of a pharmaceutical active ingredient may be masked by coating or overlaying a particle of the active ingredient with a coating or layer comprising MgSt-D. As a consequence, the surface of the taste masked particle is essentially free of the objectionable tasting pharmaceutical active ingredient. Accordingly, the taste masked particle may not interact with the taste receptors in the mouth, and release of the active ingredient from the particle may be suppressed by about five minutes in the mouth and throat.

In general, a particle of taste masked pharmaceutical active ingredient comprises a core of pharmaceutical active ingredient that is surrounded by at least one outer layer comprising MgSt-D, at least one diluent/filler, and at least one polymer. The polymer may be a pH sensitive polymer, a water insoluble polymer, or a combination thereof.

Examples of suitable pharmaceutical active ingredients were detailed above in section II(a). In general, the pharmaceutical active ingredient will have an objectionable taste, but it is also envisioned that pharmaceutical active ingredients without objectionable tastes may also be coated with the MgSt-D, diluent, and polymer coating. The pharmaceutical active ingredient typically comprises a powder with a particle size less than about 100 microns. In general, the particle will have a non-needle shape. In preferred embodiments, the pharmaceutical active ingredient may comprise a particle having an average diameter of less about 75 microns, less than about 50 microns, less than about 25 microns, or less than about 15 microns. In an exemplary embodiment, the pharmaceutical active ingredient may comprise a particle that is less than about 50 microns in diameter.

In general, the hydrophobic nature of MgSt-D provides water repellency to the outer layer that surrounds the core of pharmaceutical active ingredient. MgSt-D compositions were detailed above in section I. In preferred embodiments, the average diameter of the MgSt-D particles may be less than about 40 microns, less than about 30 microns, less than about 25 microns, less than about 20 microns, or less than about 15 microns. In another preferred embodiment, the MgSt-D particles may be may be micronized (as detailed above in section I), i.e., they are reduced to less than about 10 microns in diameter by conventional milling processes. In an exemplary embodiment, the MgSt-D particles may be less than about 30 microns in diameter.

Typically, at least one diluent/filler is included in the outer layer because the diluent/filler may facilitate formation of the taste masked particle by ensuring that the outer layer adheres to the core of active ingredient, and the diluent/filler may provide increased mechanical strength to the taste masked particle. Examples of suitable diluents/fillers were detailed above in section II(b). Preferred diluents/fillers tend to be compressible diluents, such as microcrystalline cellulose, ethylcellulose, hydroxypropyl methylcellulose, methylcellulose, polyvinylpyrrolidone, and the like. In an exemplary embodiment, the diluent/filler may be microcrystalline cellulose. Preferably, the diluent/filler is a particle having an average diameter of less than about 100 microns, less than about 75 microns, less than about 50 microns, less than about 25 microns, or less than about 15 microns. In an exemplary embodiment, the diluent/filler may comprise a particle that is less than about 50 microns in diameter.

The polymer comprising the outer layer of the taste masked particle may be a pH sensitive polymer, a water insoluble polymer, or a combination thereof. Typically, a pH sensitive polymer is an acid soluble polymer that has weakly basic functional groups (e.g., —NH2) or functional groups with tertiary ammonium, such that the polymer swells and dissolves in an acidic environment. Accordingly, the pH sensitive polymer releases the pharmaceutical active ingredient in the acid environment of the stomach (i.e., at less than about pH 5.0), but does not release the active ingredient at the pH of saliva. Examples of suitable pH sensitive polymers include aminoalkyl methacrylate copolymers, butylmethacrylate-(2-dimethylaminoethyl)methacrylate-methylmethacrylate copolymer (available as EUDRAGIT® E PO from Degussa AG, Dusseldorf, Germany), and copolymers of vinyl pyridine. Water insoluble polymers are polymers that exhibit slow swelling in water. Suitable water insoluble polymers include ethylcellulose, cellulose acetate, and polyacrylates such as methyl methacrylate, hydroxyethyl methacrylate, polymethylmethacrylate (available as ACRYL-EZE® from Colorcon Inc., West Point, Pa.).

The taste masked particles typically will comprise from about 1% to about 90% by weight of the pharmaceutical active ingredient, from about 0.1% to about 10% of MgSt-D, from about 5% to about 95% of diluentifiller, and from about 1% to about 95% of polymer, with the preferred range for polymer being from about 1% to about 70%.

The particles of taste masked pharmaceutical active ingredient may be made using a variety of techniques that are well known in the art, such as, for example, fluid bed pelletizing, direct pelletizing, spray drying, hot melt granulation, fluid bed coating, microencapsulation, and so forth. In a preferred embodiment, a pellet comprising the pharmaceutical active ingredient, MgSt-D, the diluent/filler, and the polymer may be produced using a fluid bed pelletizing process (such as, e.g., the fluid bed pelletizing system of Glaft Air Techniques, Inc., Ramsey, N.J.). Preferably, taste masked pellets of pharmaceutical active ingredient are uniformly round with an average diameter of less than about 300 microns, and have a substantially smooth surface and high density. In another preferred embodiment, a particle of taste masked pharmaceutical active ingredient may be produced by spray drying a particle of pharmaceutical active ingredient with a coating comprising MgSt-D, the diluent/filler, and the polymer or combination of polymers. Preferably, the taste masked particle produced by spray drying will be less than about 150 microns in diameter. In yet another preferred embodiment, a taste masked particle comprising the pharmaceutical active ingredient, MgSt-D, the diluent/filler, and the polymer may be produced by high shear wet granulation. Preferably, the taste masked particle produced by wet granulation will have an average diameter of less than about 300 microns.

The taste masked particles of active ingredient may be formulated into a variety of solid dosage forms such as tablets, caplets, pills, and so forth. In general, the taste masked particles will be sufficiently mechanically strong enough to withstand the blending processes and the high pressures used in tableting. The taste masked particles of active ingredient also may be formulated into other oral solid dosage forms such as orally disintegrating tablets (ODT), chewable tablets, and orally dispersible granules. For these applications, the taste masked particles may be compressed with suitable ODT placebo granules, or the taste masked particles may be simply blended with suitable ODT placebo granules. The taste masked particles in the final dosage forms are able to suppress the release of the active ingredient in the mouth and throat when the taste masked particles are in contact with saliva.

The following examples are included to demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

### Examples 1-3

### Solid-State Characterization of MgSt Hydrates

Magnesium stearate (MgSt) is widely used as a lubricant in the pharmaceutical and nutraceutical industry. MgSt is commercially available mainly in the monohydrate form (MgSt-M) or as a mixture of the monohydrate with trace amounts of the dihydrate (MgSt-D) and amorphous forms. The physicochemical properties of these two forms were examined. The following three examples detail these analyses using pure dihydrate and monohydrate forms of MgSt (derived from a vegetable source) obtained from Mallinckrodt (Hazelwood, Mo.).

### Example 1

### Particle Size and Particle Properties of the Hydrates

Particle-size distributions were determined using a laser diffraction system (series 2600, Malvern Instruments Ltd., Malvern, UK) equipped with a 63-mm lens (size range of 1.2-118 μm) and a stirred cell. Particle size, percentage of water, and concentration of neat MgSt-M and MgSt-D are presented in Table 1. The neat dihydrate had a concentration of 95.4%, and the neat monohydrate had a concentration of 92.0%. The results also revealed that the nominal mean particle size of the monohydrate was 10.6 μm, whereas the dihydrate had a nominal mean particle size of 14.3 μm. In addition, the percent bound moisture was 2.8% for MgSt-M and 5.6% for MgSt-D. Although the percent of free water was generally low, MgSt-M had 0.6%, while little or no free water was found in MgSt-D.

TABLE 1
MgSt Pseudopolymorph Particle Size and Percent Water.

| | | | | | Monohydrate | Dihydrate |
|---|---|---|---|---|---|---|
| | Monohydrate | Dihydrate | Particle size | Free water | water | water |
| Form | (%) | (%) | (μm) | (%) | (%) | (%) |
| MgSt-M | 92.0 | 0 | 10.6 | 0.6 | 2.8 | 0 |
| MgSt-D | 0 | 95.4 | 14.3 | 0 | 0 | 5.6 |

### Example 2

### Molecular Properties of the Hydrates

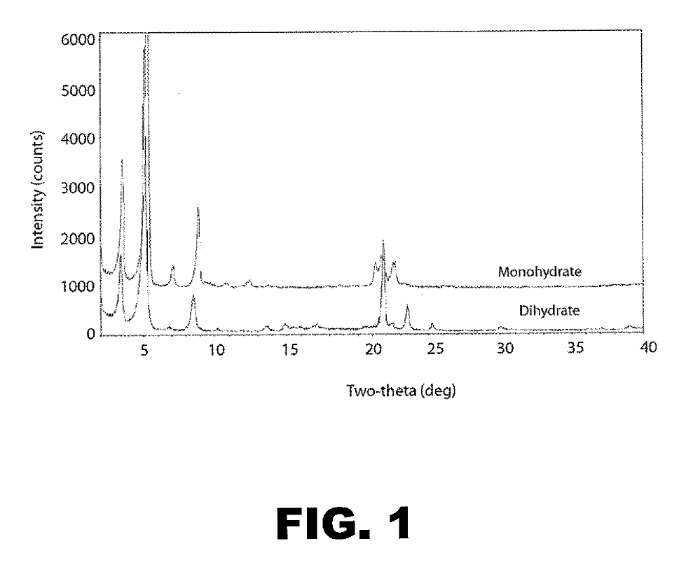

Powder X-ray diffraction patterns of the samples were measured with a Siemens D500 X-ray diffractometer over the range of 2q=2° to 40° and a 0.02° step size. FIG. 1 presents the PXRD analyses of the MgSt hydrates.

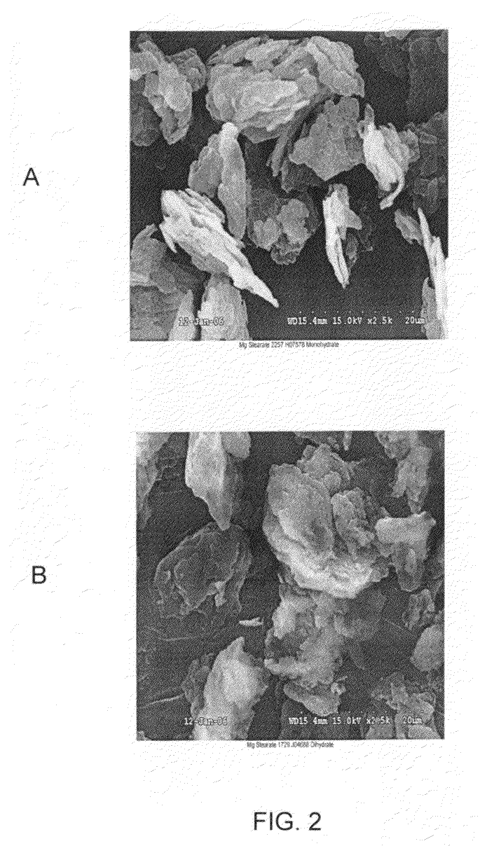

The morphologies of the samples were investigated using a scanning electron microscope (model S-4500, Hitachi, Japan). A Cressington 108 Auto/SE sputter coater was used (Cressington Scientific, Watford, UK). Images were captured with the secondary electron detector. A small portion of the powdered sample was distributed onto a conductive carbon adhesive disk on SEM stubs for SEM imaging. The specimens were sputter coated with gold-palladium to impart conductivity. The instrumental parameters were: electron beam source=W filament; accelerating voltages=15 kV; objective aperture=50 μm (aperture #3); vacuum mode=high vacuum; imaging detector(s): SE; magnification=1000×, 2500×, and 3500×; specimen tilt=0.0°; and working distance=nominal 16. FIG. 2 presents SEM micrographs of the MgSt hydrates at a magnification of 2500×.

### Example 3

### Thermal Properties of the Hydrates

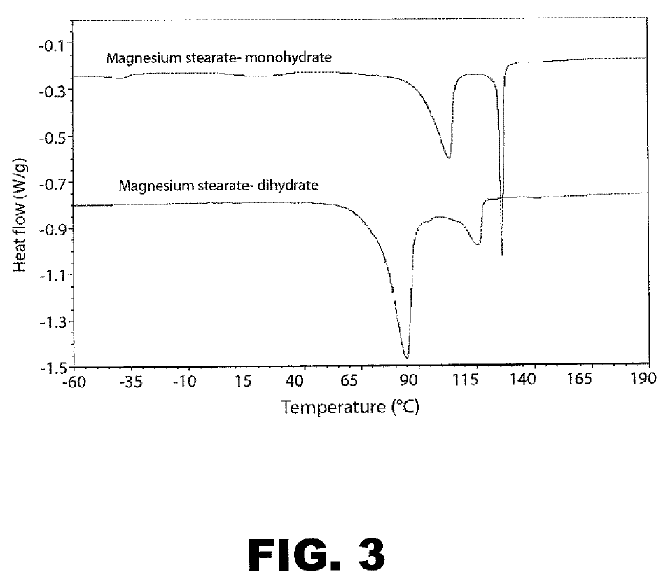

Differential scanning calorimetry (Q100 DSC, TA Instruments, New Castle, Del.) was done between temperatures from −60 to +190° C. at a heating rate of 2° C./min and a nitrogen purge of 50 mL/min. Samples (3-5 mg) were tested in crimped-aluminum pans, and an empty pan was used as a reference. The temperature axis and cell constant of DSC were previously calibrated with pure standard of indium. Data acquisition and analysis were conducted using TA Instruments software. DSC profiles of the MgSt hydrates are presented in FIG. 3.

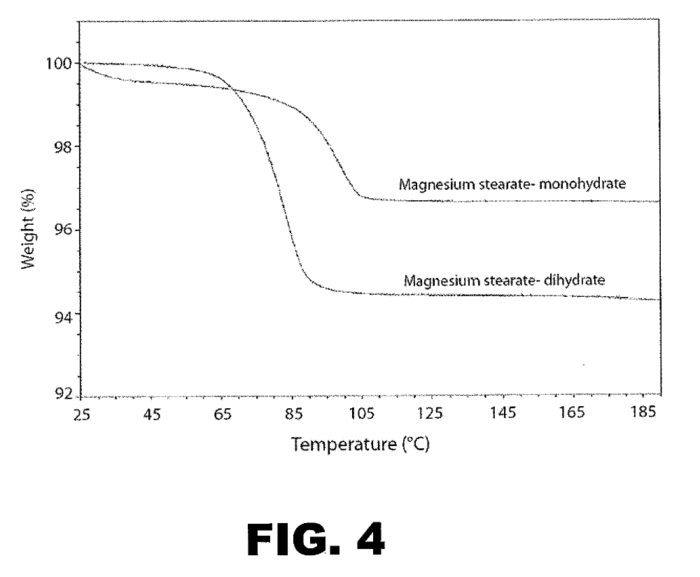

Thermal gravimetric analysis (Q50 TGA, TA Instruments) was carried out from 25 to 190° C. at a heating rate of 5° C./min. Data acquisition and analysis were conducted using TA Instruments software. FIG. 4 presents the TGA of MgSt-M and MgSt-D.

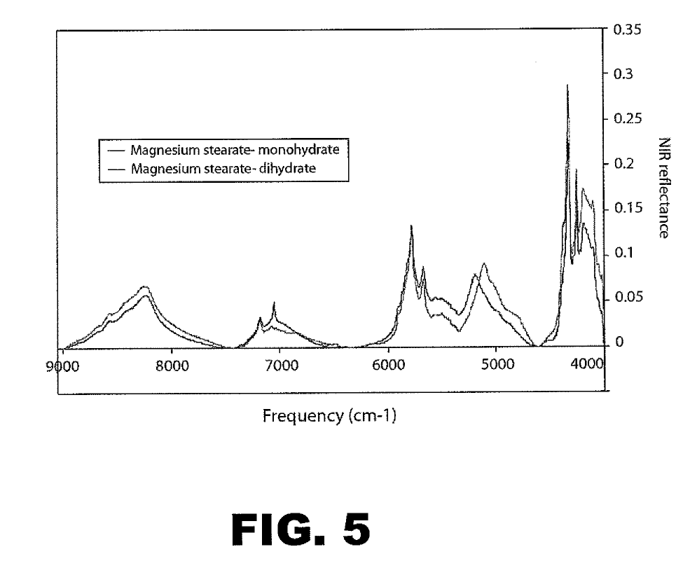

Near infrared spectroscopy analysis (NIR analyzer, Thermo Fisher Scientific, Waltham, Mass.) was conducted using standard mixtures having known compositions of MgSt monohydrate and dihydrate prepared from 92.0% MgSt-M and 95.4% MgSt-D stock material. The NIR spectra of the MgSt hydrates are shown in FIG. 5.

Conclusions. The physicochemical analysis presented in Examples 1-3 using scanning electron microscopy, powder X-ray diffraction, near-infrared spectroscopy, differential scanning calorimetry, particle-size analysis, and thermogravimetry revealed that there are discernable differences between the MgSt monohydrate and MgSt dihydrate.

### Examples 4-8

### Influences of MgSt Hydrates on Blends and Tablets

The influence of MgSt on powder lubrication and finished solid-dose products has presented significant challenges to drug manufacturing, including poor production efficiency and variability in drug disintegration and dissolution. The variation of crystalline states and their amounts in MgSt products could affect consistency in powder blending and the compressibility and quality of the resulting tablets. These examples examine the influence of MgSt hydrates on blends and tablets using ternary systems comprising an active pharmaceutical ingredient with different diluent mixes (i.e., a plastically deformable-brittle diluent mix or a plastically deformable-plastically deformable diluent mix). Various ratios of diluents were used to justifiably rule out any ingredient bias that could be attributed to lubricant affinity to one diluent system. The influence of the pseudopolymorphic MgSt-M and MgSt-D on blends was profiled in real time with in-line thermal effusivity sensors during blending and lubrication steps and with an instrumented tablet press during compression. The generation of different blends is detailed in Example 4, and the difference blends are characterized in Examples 5-8.

### Example 4

### Production of Blends and Tablets

The active pharmaceutical ingredient was acetaminophen, USP (APAP, Mallinckrodt) and the diluents were microcrystalline cellulose (MCC; Avicel PH 101, 102, FMC Biopolymer, Philadelphia, Pa.); dibasic calcium phosphate, anhydrous (DCP, Encompress, JRS Pharma, Patterson, N.Y.); and lactose monohydrate, spray-dried (LAC, Spectrum Chemicals, NJ). All materials were used as received and delumped before mixing.

Two ratios of binary diluents were used (75:25 and 50:50) for each of MCC:DCP and MCC:LAC. These binary diluents constituted desirable solid dosage formulation systems in that the physical characteristics are unique. In the case of the MCC:DCP system, the physical interaction between a plastically deformable material (MCC) and an abrasively brittle material (DCP) with distinct particle-particle shapes was deemed informative. Moreover, using two grades of MCC with different particle sizes (Avicel PH 101 had a nominal mean particle size of 50 μm, and Avicel PH 102 had a nominal mean particle size of 100 μm) could present additional valuable information through their blending behavior. With respect to the MCC:LAC system, two plastically deformable diluents with distinct particle-particle morphology would be another opportunity to elucidate the influence of MgSt in such widely used pharmaceutical combinations.

APAP was used at concentrations of 1.25, 2.5, and 5.0% w/w. MgSt was used at concentrations of 0.3, 0.5, and 1.0% w/w. The experimental design was a modified Plackett-Burman fractional factorial having two levels with two center points. Eleven batches, each at 10-kg batch size, were blended in a 1-ft³ twin-shell blender (Patterson-Kelley, Stroudsburg, Pa.). This fractionalization allowed for a reduction of input variables or factors with the benefit of identifying the key factor variables that affected product quality. The design also enabled the evaluation of main effects aliased with two-way interactions. Tables 2 and 3 show the designs and independent variables (factors). Results from the experimental design provided information for optimization of the study (see Table 2). Subsequently, six optimization batches were processed to substantiate the preliminary findings from the 11-batch runs (see Table 3). The dependent variables (responses) included ejection force and total compression force (precompression and main compression forces).

TABLE 2
Experimental Design.

| | | | Lube | | | |
|---|---|---|---|---|---|---|
| | MCC | | Time | | | MCC/ |
| Batch No. | (μm) | MgSt Type | (min) | % MgSt | APAP (%) | DCP ratio |
| 1 | 50 | MgSt-M | 10 | 0.3 | 5.0 | 75/25 |
| 2 | 50 | MgSt-D | 4 | 0.3 | 5.0 | 50/50 |
| 3 | 100 | MgSt-M | 4 | 0.3 | 2.5 | 50/50 |
| 4 | 100 | MgSt-D | 10 | 0.3 | 2.5 | 75/25 |
| 5 | 100 | MgSt-M | 7 | 0.5 | 1.25 | 50/50 |
| 6 | 50 | MgSt-D | 7 | 0.5 | 1.25 | 50/50 |
| 7 | 50 | MgSt-M | 4 | 1.0 | 2.5 | 75/25 |
| 8 | 100 | MgSt-M | 10 | 1.0 | 1.25 | 75/25 |
| 9 | 100 | MgSt-M | 4 | 1.0 | 1.25 | 50/50 |
| 10 | 50 | MgSt-D | 10 | 0.3 | 5.0 | 75/25 |
| 11 | 50 | MgSt-D | 4 | 1.0 | 2.5 | 75/25 |

TABLE 3
Optimized Design.

| Batch | MgSt | Lube Time | | APAP | MCC_{50 μm}/LAC |
|---|---|---|---|---|---|
| No. | Type | (min) | % MgSt | (%) | ratio |
| 12 | MgSt-M | 4 | 1.0 | 1.25 | 75/25 |
| 13 | MgSt-M | 2, 4, 8, 12, 16 | 1.0 | 1.25 | 75/25 |
| 14 | MgSt-D | 4 | 1.0 | 1.25 | 75/25 |
| 15 | MgSt-D | 2, 4, 8, 12, 16 | 1.0 | 1.25 | 75/25 |
| 16 | MgSt-M | 4 | 1.0 | 1.25 | 50/50 |
| 17 | MgSt-D | 4 | 1.0 | 1.25 | 50/50 |

Prelubrication blend uniformity was predicted using multiple effusivity sensors fitted to the blender as described by Okoye et al. (2006, ISPE News Magazine 3(3):4-8). Prelubrication and postlubrication blend uniformity samples were collected using a sampling thief (Globe Pharma, New Brunswick, N.J.) for comparative analysis. Blend samples were analyzed for the APAP assay with an internally validated high-performance liquid chromatography (HPLC) method.

The blends were compressed using a 10-station instrumented tablet press (Natoli Engineering, St Louis, Mo.) with 0.4375-in. standard round, concave tooling. All tablets were compressed to target hardness of 8.0 kp using a hardness tester (Pharmatest, Piscataway, N.J.), and target weight of 500 mg was measured using a bench scale (Sartorius, New York, N.Y.). Tablet press speed was maintained at 17 rpm. Tablet friability limit was set at not more than (NMT) 0.8% using a friability tester (Pharmatest).

Lubricant performance and influence on the tablets' physical attributes were evaluated on the basis of the main compression force, precompression force, ejection force, and tablet knock-off using a real-time data acquisition tool (Natoli Engineering, St. Louis, Mo.). In vitro dissolution studies were conducted according to USP Method, and a similarity factor, f₂, was derived for comparative analysis. Data analysis was conducted using a statistical tool (“Minitab,” Minitab Inc., State College, Pa.).

### Example 5

### Predicted Blend Homogeneity and Assay

A baseline run was conducted using neat microcrystalline cellulose, NF (MCC) to enable the effusivity sensors to predict homogeneity via in-line and real-time measurements. The placebo material was blended for a specified duration, and the synchronization pulse with baseline was established for the effusivity sensors.

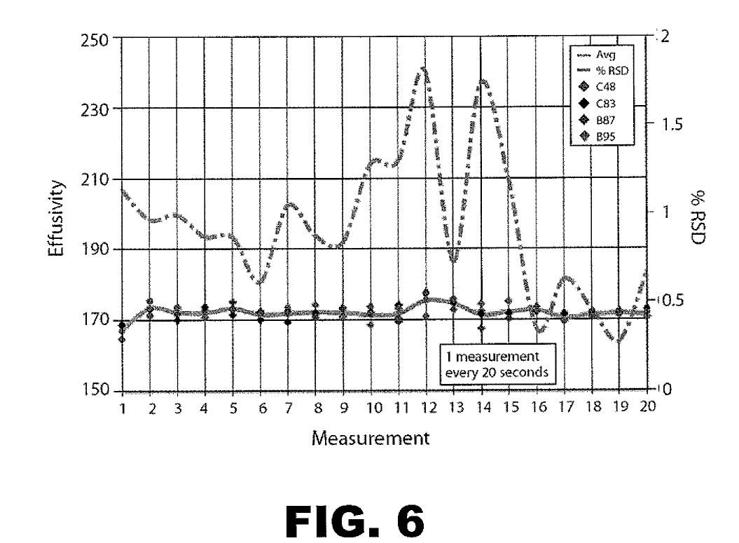

The prelubrication homogeneity of the blends was determined on the basis of real-time analysis conducted with Effusivity Sensor Package software (ESP, Mathis Instruments, Fredericton, Canada). The system synchronization enabled the sensors to dynamically obtain a real-time data stream from the rotating blender (see FIG. 6). Effusivity sensors monitor the blending of powder particles on the basis of the heat-transfer properties of the composite powder mixture. An explanation for this sensitivity to material blending or mixing is related to the ability of these powder particles or ingredients to mix in various proportions as they attend homogeneity. This is analogous to a move from “divergence” toward “convergence” or uniformity. The selection of any empirical uniformity value may depend on several factors, including the nature of materials, type of process, and individual effusivity values of the ingredients. It is well established that low relative standard deviation (RSD) in a blending process signifies uniformity in mixing or blending. End-point prediction with effusivity was based on the combination of stable average effusivity and a decaying RSD.

Blend samples were collected using a sample thief from each batch at the end of prelubrication and postlubrication blending. Blend samples of about two times the unit-dose (500 mg×2=1000 mg) were tested based on an internally validated HPLC method for APAP. The mobile phase was a mixture of methanol and water with a flow rate of 1.0 mL/min and detection at 280 nm.

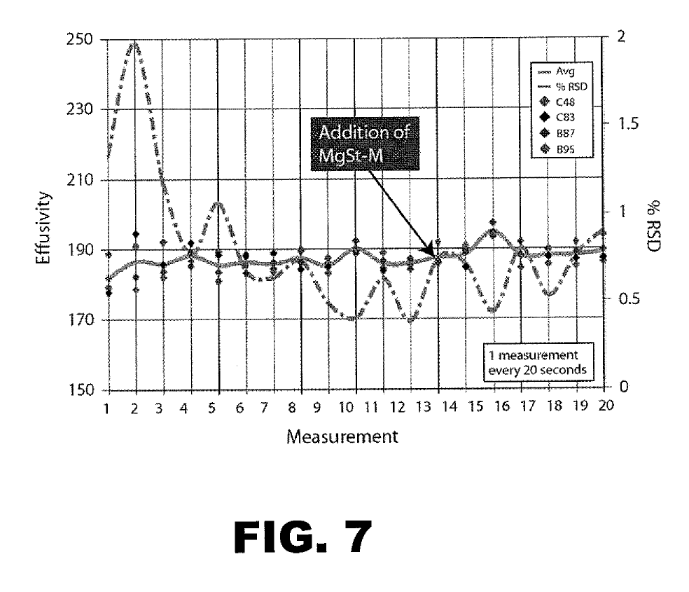

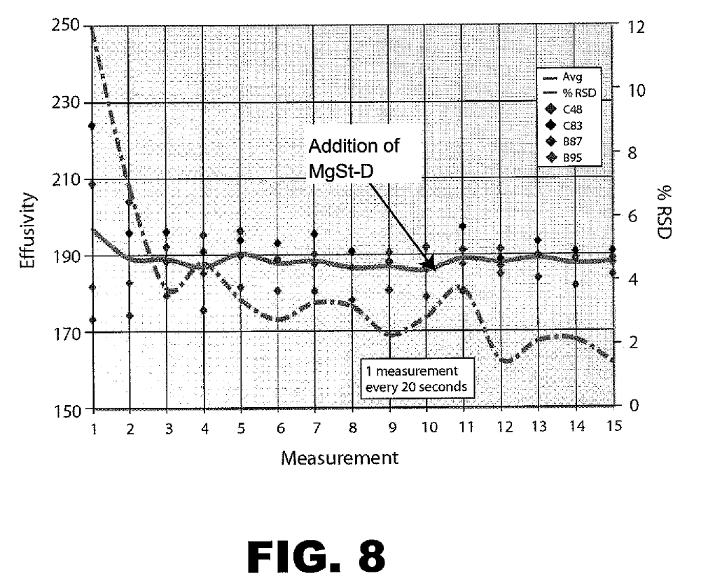

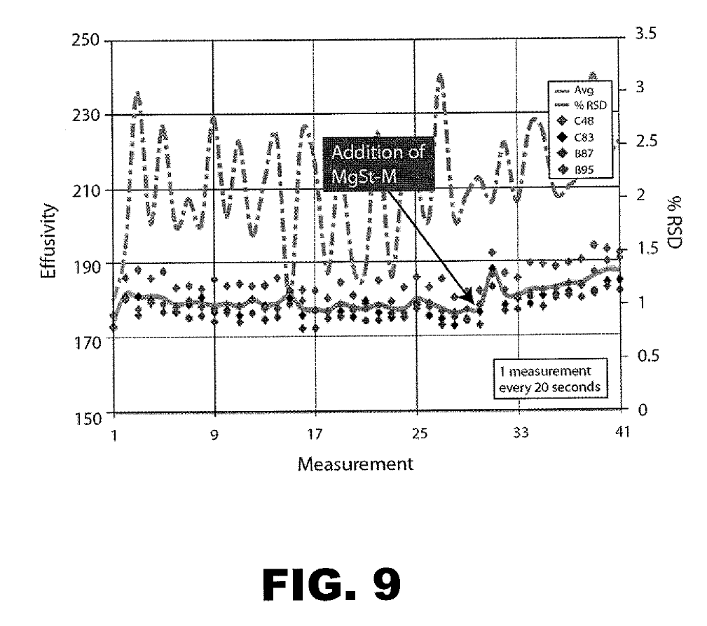

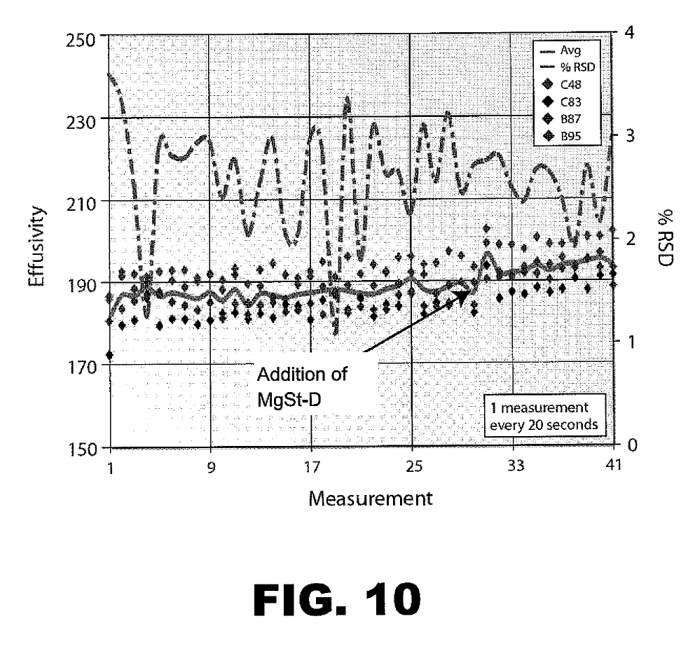

The profile of the baseline run with neat MCC is depicted in FIG. 6. Results show an average effusivity for the placebo material of 171 Ws^{0.5}/m²K (n=20), with RSD=0.7%. FIGS. 7 (Batch 7) and 8 (Batch 11) show the effusivity profiles for ternary blends of MCC, DCP (75:25), and 2.5% w/w APAP lubricated with 1.0% w/w MgSt-M and MgSt-D, respectively. FIGS. 9 (Batch 12) and 10 (Batch 14) show the effusivity profiles for ternary blends of MCC, LAC (75:25), and 1.25% w/w APAP lubricated with 1.0% w/w MgSt-M and MgSt-D, respectively.

Table 4 shows the results of the physical and chemical testing for the blends. Blend results indicate that the prelubrication end-points as predicted by effusivity sensors gave good correlation to the blend assay from HPLC analysis. Blend uniformity results for Batch 12, after 4 min of lubrication with MgSt-M, however, show a mean blend uniformity assay of 109.0%, with a failing RSD of 23.5%. Conversely, Batch 14 lubricated with MgSt-D shows an acceptable mean blend uniformity assay of 94.8% with an RSD of 1.6%.

TABLE 4
Blend Uniformity and Effusivity Results.

| | Batch 7 | Batch 11 | Batch 12 | Batch 14 |
|---|---|---|---|---|
| Prelube blend unifomity | 100.3 | 99.3 | 98.8 | 100.8 |
| assay | | | | |
| % RSD on assay | 1.5 | 2.0 | 1.2 | 3.1 |
| Postlube blend | 97.5 | 97.0 | 109.0 | 94.8 |
| uniformity assay | | | | |
| % RSD on post lube | 1.7 | 1.4 | 23.5* | 1.6 |
| at 4 min | | | | |
| Prelube end-point | 188.4 | 188.5 | 178.0 | 188.0 |
| effusivity | | | | |
| % RSD in prelube | 2.3 | 2.2 | 2.0 | 2.5 |
| effusivity | | | | |
| % MgSt used | 1.0 | 1.0 | 1.0 | 1.0 |
| MgSt type | MgSt-M | MgSt-D | MgSt-M | MgSt-D |
| MCC_{50 μM}:DCP ratio | 75:25 | 75:25 | | |
| MCC_{50 μM}:LAC ratio | | | 75:25 | 75:25 |
| % APAP (w/w) | 2.50 | 2.50 | 1.25 | 1.25 |

*Batch 12 lubricated with 1.0% MgSt-M failed on RSD.

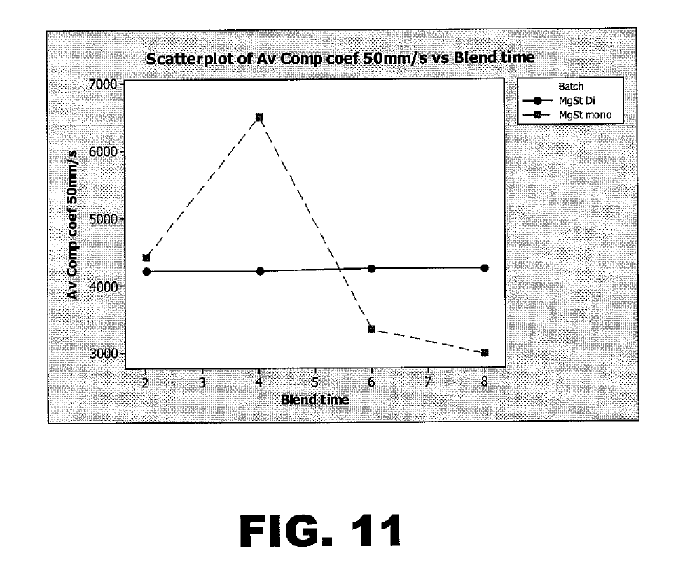

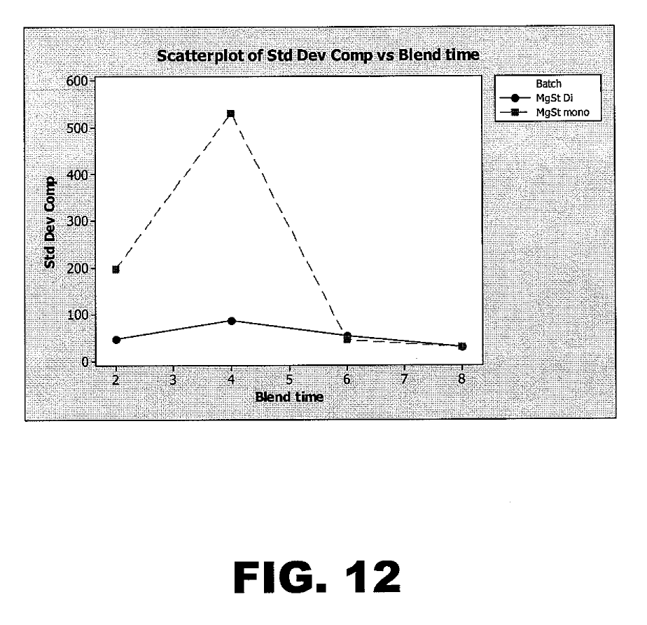

Scatterplot analysis of the average compression coefficient 50 mm/s as a function of blend (i.e., lubrication) time revealed that dihydrate blends differed dramatically from monohydrate blends at 4 min (see FIG. 11). Similarly, scatterplot analysis of the compression standard deviation vs. blend time showed dihydrate blends differed dramatically from monohydrate blends at 4 min (see FIG. 12).

### Example 6

### The Influence of Lubrication and MgSt Type on Blend Integrity

The influence of MgSt type and concentration on effusivity was analyzed using one-way analysis of variance (ANOVA). Analysis of the change (delta) in average effusivity between prelubrication and postlubrication blends containing MgSt-M and MgSt-D, using Tukey's paired comparison at 95% confidence limit, shows statistical significance of p<0.05 (see Table 5). The pair wise comparison is indicative of the differing influence attributable to the distinct hydrate forms of the lubricant.

TABLE 5
The Influence of MgSt Type on Blend Integrity.

| | | | Factors | | Responses | | |
|---|---|---|---|---|---|---|---|
| | | Diluent | MgSt | | Delta effusivity* | Total comp | Eject force |
| Batch | Diluent system | ratio | type | % MgSt | (Ws^{0.5}/m²K) | force (kN) | (N) |
| 1 | MCC:DCP | 75:25 | Mono | 0.3 | 10 | 12 | 11 |
| 7 | MCC:DCP | 75:25 | Mono | 1.0 | 7 | 13.1 | 11 |
| 10 | MCC:DCP | 75:25 | Di | 0.3 | 3 | 12.1 | 10 |
| 11 | MCC:DCP | 75:25 | Di | 1.0 | 3 | 11.2 | 9 |
| 12 | MCC:LAC | 75:25 | Mono | 1.0 | 10 | 10.3 | 8.6 |
| 14 | MCC:LAC | 75:25 | Di | 1.0 | 6 | 9.3 | 6.7 |
| 16 | MCC:LAC | 50:50 | Mono | 1.0 | 16 | | |
| 17 | MCC:LAC | 50:50 | Di | 1.0 | 10 | | |

*Delta effusivity is the change in average effusivity between post- and pre-lubrication. All batches were lubricated for 4 min.

Results from the blending studies, as profiled by the in-line effusivity sensors, also showed that when ternary systems containing MCC-DCP and MCC-LAC as diluents were lubricated with MgSt-M and MgSt-D, the delta effusivity values were higher for the blends containing MgSt-M. (Compare Batch 7 and Batch 12 versus Batch 11 and Batch 14 in Table 5). These results indicate the ternary systems containing MgSt-D showed less degree of densification for both MCC-LAC and MCC-DCP diluent systems before and after lubrication.

Similarly, with the same 75:25 diluent ratio, MCC-DCP blends lubricated with MgSt-M exhibited 2-3 times more densification than MgSt-D (Batch 7 versus Batch 11). Also MCC-LAC blends with a 75:25 ratio, when lubricated with MgSt-M, showed about 1.6 times more densification than blends with MgSt-D (Batch 12 versus Batch 14). Moreover, within the MCC-LAC diluent system, the 50:50 diluent ratio tended to show higher delta effusivity than the 75:25 ratio. (Batch 12 versus Batch 16, and Batch 14 versus Batch 17). This result could be attributed to the increasing contribution of lactose in the formulation, particle-particle interaction, and diluent-type sensitivity to the influence of MgSt. Although the mechanism of the densification may not be fully understood, it is believed that the finer particles of the lubricant tend to displace the air pockets between larger particles and occupy the interstices with a resultant more densely packed powder mixture. Such particulate packing, presumably a result of MgSt addition, could disturb the established blend uniformity.

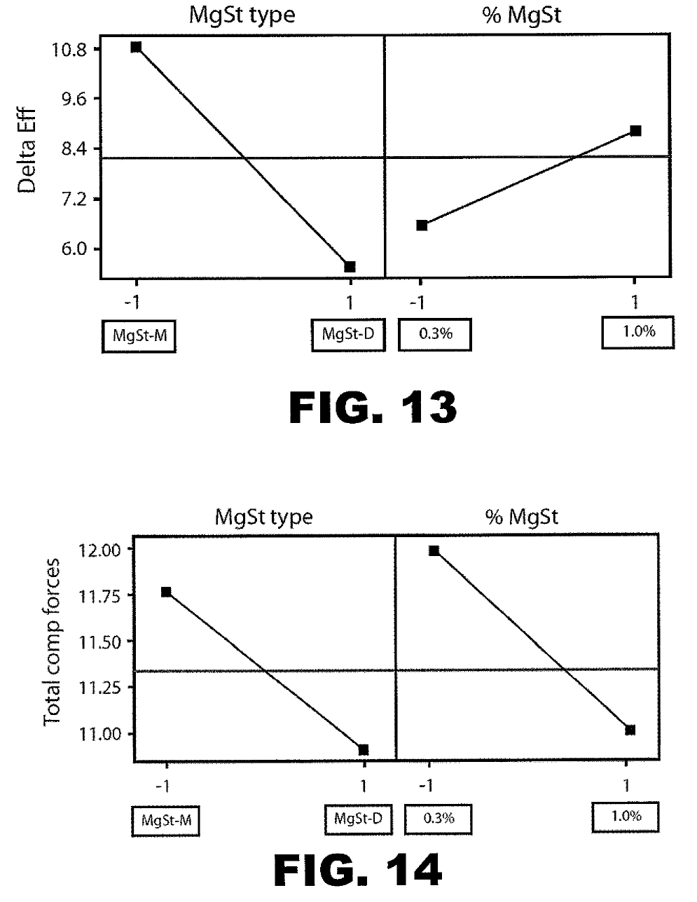

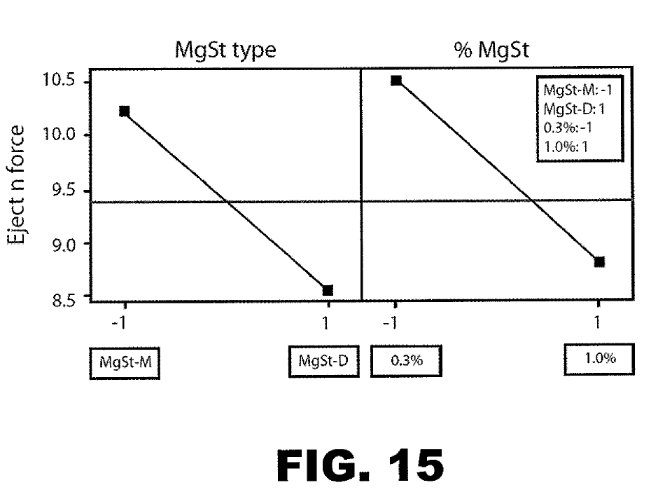

FIG. 13 shows that the delta on average effusivity was greater for MgSt-M than for MgSt-D. In addition, the results indicate that the average total compression forces and ejection force were lower for MgSt-D than the MgSt-M (see Table 6 and FIGS. 14 and 15).

### Example 7

### Physical Attributes and Content Uniformity of the Tablets

Compression of the batches was conducted using a 10-station instrumented press (Natoli Engineering). Compression parameters were monitored based on constant (target) tablet weight (500 mg) and hardness (8 kp). Based on the fact that MgSt type, percentage of MgSt, and lubrication time differed in the batches, the effects of these variables on precompression force, main compression force, ejection force, knock-off force, and tablet friability were monitored or measured to evaluate the level of such influence. Additional influence was also expected from the differing percentage of APAP and diluents. An attempt was made to statistically analyze such influence to understand the main effects and interactions.

Using a stratified sampling method, tablets were collected at intervals during the compression runs. Content uniformity was conducted using an internally validated HPLC method for APAP. Mean assay and % RSD for 10 tablets were determined.

The data in Table 6 show the compression batches containing MCC-DCP and MCC-LAC binary diluents systems. Results show that except for Batch 8, all batches gave acceptable results for content uniformity. The mean assay for 10 tablets for Batch 8 was 96.9%. The % RSD was 7.9, however, which is much higher than the acceptable limit. Batch 8 was lubricated with 1.0% of MgSt-M for 10 min. This result implies that an extended period of lubrication could affect the tablets' content uniformity.

TABLE 6
Physical and Chemical Results for Tablets.

| | Batch Number | | | | | | | | | | | | |
|---|---|---|---|---|---|---|---|---|---|---|---|---|---|
| | 1 | 2 | 3 | 4 | 5 | 6 | 7 | 8 | 9 | 10 | 11 | 12 | 14 |
| Average tablet weight | 493.9 | 508.0 | 504.4 | 491.0 | 499.4 | 496.1 | 507.7 | 502.4 | 497.5 | 497.4 | 491.2 | 497.0 | 497.0 |
| (mg) | | | | | | | | | | | | | |
| RSD on tablet weight | 0.4 | 0.6 | 1.3 | 0.4 | 1.3 | 1.6 | 0.5 | 0.9 | 0.5 | 0.3 | 0.9 | 0.5 | 0.6 |
| (n = 10) | | | | | | | | | | | | | |
| Average tablet hardness | 8.4 | 8.1 | 8.0 | 8.0 | 8.1 | 8.1 | 8.1 | 8.5 | 8.3 | 8.5 | 8.3 | 8.2 | 8.1 |
| (kp) | | | | | | | | | | | | | |
| MgSt type | Mono | Di | Mono | Di | Mono | Di | Mono | Mono | Di | Di | Di | Mono | Di |
| MCC:DCP ratio | 75:25 | 50:50 | 50:50 | 75:25 | 50:50 | 50:50 | 75:25 | 75:25 | 50:50 | 75:25 | 75:25 | | |
| MCC:LAC ratio | | | | | | | | | | | | 75:25 | 75:25 |
| Lubrication time (min) | 10 | 4 | 4 | 10 | 7 | 7 | 4 | 10 | 4 | 10 | 4 | 4 | 4 |
| % MgSt | 0.3 | 0.3 | 0.3 | 0.3 | 0.5 | 0.5 | 1.0 | 1.0 | 1.0 | 0.3 | 1.0 | 1.0 | 1.0 |
| MCC particle size (μm) | 50 | 50 | 100 | 100 | 100 | 50 | 50 | 100 | 100 | 50 | 50 | 50 | 50 |
| APAP (%) | 5.0 | 5.0 | 2.5 | 2.5 | 1.25 | 1.25 | 2.5 | 1.25 | 1.25 | 5.0 | 2.5 | 1.25 | 1.25 |
| Precomp force (kN) | 3.75 | 2.81 | 3.12 | 3.87 | 3.06 | 4.07 | 4.12 | 4.67 | 3.67 | 3.77 | 3.59 | 4.42 | 4.40 |
| Main compression (kN) | 8.24 | 12.9 | 13.61 | 7.87 | 11.71 | 12.6 | 8.99 | 11.47 | 18.4 | 8.36 | 7.63 | 5.86 | 4.92 |
| Ejection (N) | 10.6 | 24.3 | 16.4 | 10.7 | 14.3 | 15.9 | 10.9 | 13.0 | 19.4 | 10.4 | 9.3 | 8.6 | 6.7 |
| Total comp force (kN) | 11.99 | 15.71 | 16.73 | 11.74 | 14.77 | 16.67 | 13.11 | 16.14 | 22.07 | 12.13 | 11.22 | 10.3 | 9.3 |
| Mean content uniformity | 101.6 | 97.2 | 103.9 | 104.4 | 99.8 | 97.2 | 99.1 | 96.9 | 98.5 | 98.3 | 96 | 99.1 | 98.2 |
| (%) | | | | | | | | | | | | | |
| % RSD on content | 2.1 | 4.3 | 1.7 | 3.0 | 2.4 | 1.9 | 2.4 | 7.9* | 1.9 | 3.2 | 1.7 | 1.5 | 2.0 |
| uniformity | | | | | | | | | | | | | |

*Batch 8 lubricated with 1.0% MgSt-M failed on RSD.

The tablet characteristics shown in Table 6 depict some distinct effects in the total compression forces, ejection force, and tablet knock-off between the blends lubricated with different pseudopolymorphic forms of MgSt. These differences in tableting forces appear to be evident under similar formulations and with preset target ranges for tablet weight and hardness. So long as the preblend components of the formula are comparable, the anticipated variables would include percentage of MgSt and duration of lubrication. These two variables tend to influence the compressibility, tablet ejection, and knock-off. The efficiency of a lubricant during a tableting operation hinges on its ability to facilitate tablet release postcompression. The amount of such lubricants, however, combined with the duration of lubrication, often influence the forces acting on the upper and lower punches.

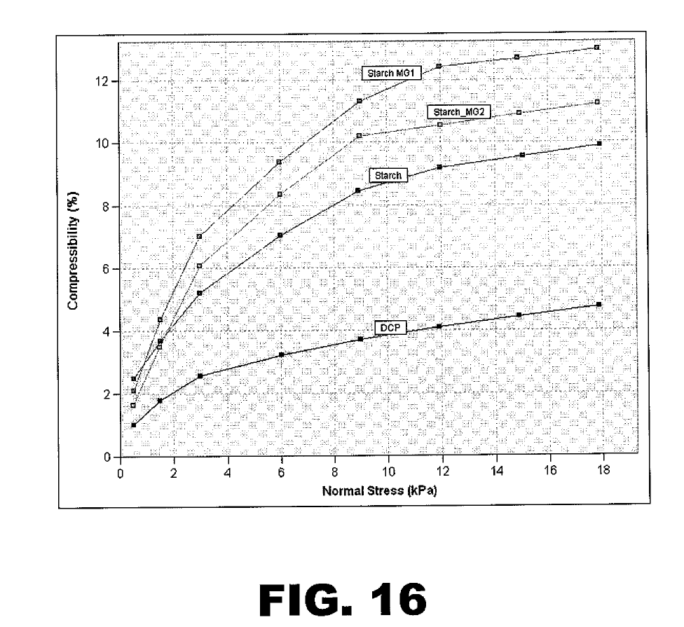

A powder rheometer (FT4, Freeman Technology, Worcestershire, UK) was used to measure compressibility of tablets that had starch as a diluent. FIG. 16 shows that tablets with either MgSt hydrate had greater compressibility than those without.

Based on a Plackett-Burman design, % MgSt, MCC-DCP ratio, MSS particle size, % APAP, and lubrication time were evaluated for their influence on ejection force and total compression force. Using the method of least squares, regression models were developed for the total force (precompression and main compression forces) and the ejection force to elucidate the influence of the lubricants on the compression process.

Table 7 shows the regression analysis for the ejection force. Based on the tablet physical results, the diluent ratio had the greatest influence on ejection force (p<0.005). The data also showed that the % API and % MgSt in the formulation had second- and third-highest influence on tablet ejection based on the coefficient at p<0.005. Overall, R² (indicating the linearity of the regression) was 0.9250, suggesting that the selected model design was appropriate.

TABLE 7
Regression Analysis for Ejection Force.

| Predictor | Coefficient | SE coefficient | T | P |
|---|---|---|---|---|
| Constant | 16.352 | 0.880 | 18.470 | 0.000 |
| % MgSt | 4.477 | 1.400 | 3.200 | 0.024 |
| Lube time | 2.775 | 1.060 | 2.610 | 0.048 |
| Particle size | 1.837 | 0.665 | 2.760 | 0.040 |
| Diluent ratio | −6.537 | 1.007 | −6.490 | 0.001 |
| % APAP | 5.858 | 1.545 | 3.790 | 0.013 |
| S = 1.1813 | R² = 92.5% | R² (adj) = 83.6% | | |

In addition, the regression model for the total forces (precompression and main-compression forces) as depicted in Table 8 shows that the diluent ratio also had the highest influence on combined compression forces (p<0.005). The second and third highest-ranking responses, based on the coefficient at p<0.005, were the percentage of MgSt and the type of MCC, respectively. The model shows a linearity, R²=0.9110. These regression models show that the influence of the diluent ratio, percentage of MgSt, percentage of API, and type of MCC, if held constant, could offer some insights into the subtle characteristics of other factors such as the type of MgSt and the duration of lubrication. As such, an optimized design was constructed to keep these factors the same and minimize their influence to fully elucidate the presence (or absence) of influence of differing MgSt hydrates.

TABLE 8
Regression Analysis for Total Force (Precompression and Main).

| Predictor | Coefficient | SE coefficient | T | P |
|---|---|---|---|---|
| Constant | 1622.480 | 66.630 | 24.350 | 0.000 |
| % MgSt | 313.800 | 105.400 | 2.980 | 0.031 |
| Lube time | 147.240 | 79.960 | 1.840 | 0.125 |
| Particle size | 176.950 | 50.070 | 3.530 | 0.017 |
| Diluent ratio | −355.840 | 75.750 | −4.700 | 0.005 |
| % APAP | 216.200 | 116.200 | 1.860 | 0.122 |
| S = 136.5 | R² = 91.1% | R² (adj) = 80.3% | | |

Two experiments were conducted using a binary diluent system of MCC (50 μm particle size) and LAC at the ratio of 75:25, with APAP as the active ingredient at 1.25% w/w concentration. With the level of MgSt at 1.0% w/w, the influence of lubrication on blend uniformity assay was monitored at 2-, 4-, 8-, 12-, and 16-min time points. Results show that Batch 13 lubricated with MgSt-M gave failing % RSD on blend uniformity assay at 2-min (36.0%) and 4-min (8.1%) time points (see Table 9). Results at the 8-, 12-, and 16-min time points, however, were acceptable. For Batch 15 lubricated with MgSt-D, the results at all the time points were acceptable. Although no reason was found for the failed results for Batch 13, the influence of the MgSt type on a uniformity blend could not be ruled out.

TABLE 9
Postlubrication of MCC-LAC Binary Diluent Systems.

| | Batch 13 | Batch 15 |
|---|---|---|
| | (MgSt-M) | (MgSt-D) |
| Prelube | | |
| Mean | 92.6 | 98.8 |
| RSD | 3.2 | 2.7 |
| Postlube blend uniformity (%) | | |
| 2 min | | |
| Mean | 98.7 | 94.9 |
| RSD | 36.08 | 1.7 |
| 4 min | | |
| Mean | 92.0 | 95.5 |
| RSD | 8.18 | 2.0 |
| 8 min | | |
| Mean | 95.6 | 98.1 |
| RSD | 2.7 | 3.8 |
| 12 min | | |
| Mean | 96.8 | 95.7 |
| RSD | 3.3 | 1.5 |
| 16 min | | |
| Mean | 97.1 | 97.1 |
| RSD | 0.9 | 2.1 |

* Batch 13 failed RDS at 2 and 4 min lubrication.

### Example 8

### In Vitro Dissolution of the Tablets

In vitro dissolution studies were performed using an USP Type 2 dissolution apparatus at 50 rpm. The dissolution media consisted of 900 mL degassed purified water, USP, maintained at 37° C.±0.5° C. A 5-mL aliquot was withdrawn at intervals of 5, 10, 15, and 30 min. Drug content was determined by HPLC at 280 nm. All dissolution tests were conducted in triplicate. The similarity was determined by the model independent approach using a similarity factor (f₂) as described in the FDA Guidance for Industry: Dissolution Testing of Immediate Release Solid Oral Dosage Forms (1997; FDA, Rockville, Md.). The similarity factor (f₂) is defined as follows:

$${f}_{2} = 50 \times log [ {[ 1 + ( \frac{1}{n} ) {\sum }_{t = 1}^{n} {( {R}_{t} - {T}_{t} )}^{2} ]}^{- 0.5} \times 100 ]$$

in which Rₜ and Tₜ are the average percentage of drug dissolved at each sampling time for reference (R) and the test (T) preparations, respectively, and n is the number of samples. An f₂ value between 50 and 100 suggests that the two dissolution profiles are similar.

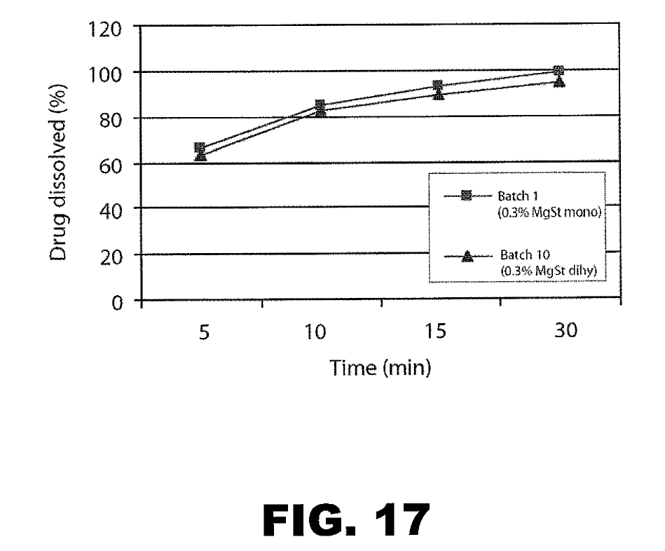

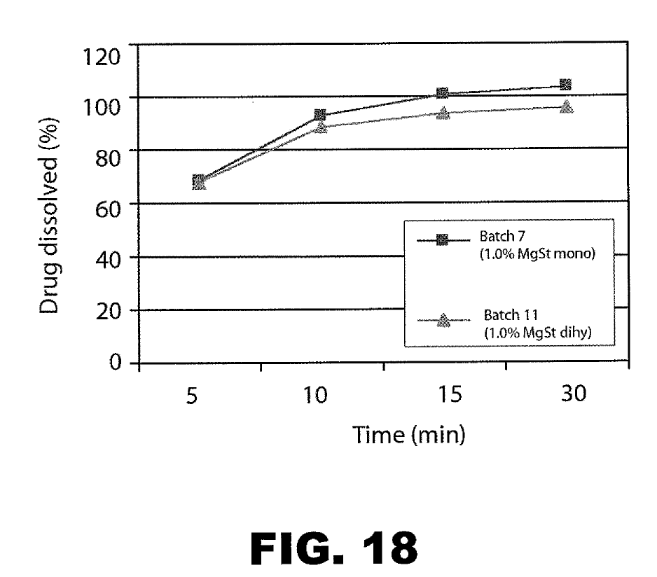

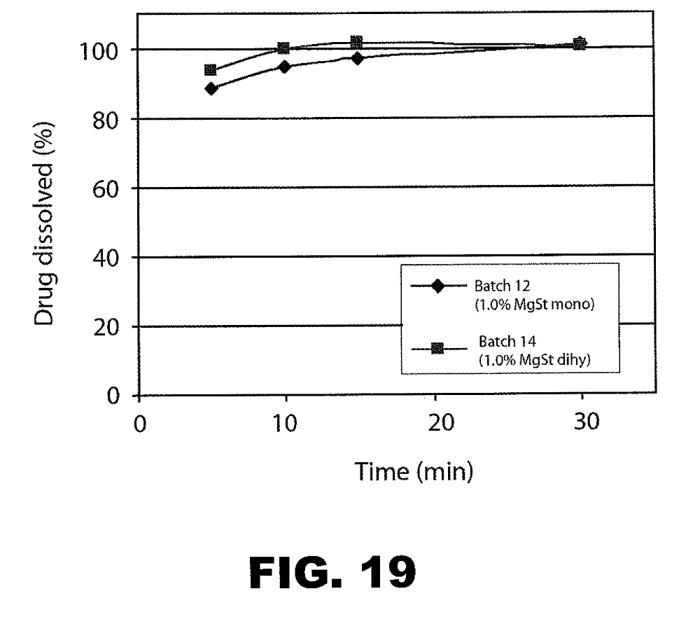

In vitro dissolution was conducted on six tablets from each of batches 1, 7, 10, 11, 12, and 14. These batches were lubricated with different amounts of MgSt-M and MgSt-D ranging from 0.3% to 1.0%. FIG. 17 shows results from Batch 1 and Batch 10 lubricated for 10 min with 0.3% MgSt-M and MgSt-D, respectively. FIG. 18 shows results of Batch 7 and Batch 11 lubricated for 4 min with 1.0% MgSt-M and MgSt-D, respectively. And FIG. 19 shows results from Batch 12 and Batch 14 lubricated for 4 min with 1.0% MgSt-M and MgSt-D, respectively. Results of the in vitro dissolution suggest that all batches met the criteria for similarity (f₂ between 50 and 100) as stipulated in the FDA manual (see FIGS. 14-16 and Table 10). These results indicate that finished products containing the dihydrate polymorph could provide comparable quality to those containing the monohydrate form.

TABLE 10
Similarity factor (f₂) for (MCC-DCP) Tablets with Different
MgSt Types.

| | Drug dissolved (%) | | | |
|---|---|---|---|---|
| | Batch 1 | Batch 10 | Batch 7 | Batch 11 |
| Time (min) | (MgSt-M) | (MgSt-D) | (MgSt-M) | (MgSt-D) |
| 5 | 66.2 | 63.6 | 68.8 | 68.2 |
| 10 | 85.3 | 82.6 | 93 | 88.3 |
| 15 | 92.8 | 89.8 | 100.5 | 93.6 |
| 30 | 99.2 | 95 | 103.6 | 95.8 |

| | |
|---|---|
| f₂comparison = 74 | f₂comparison = 62 |

Conclusions. The lubrication of direct-compressible blends with the hydrates of MgSt has presented evidence of differences in the effects these hydrates could have on blend homogeneity and tablet compression. In-line effusivity sensors predicted blend uniformity in all prelubrication blends down to 1.25% w/w of active pharmaceutical ingredient in the formulations. In addition, the in-line effusivity sensors suggested that lubricating blends with the monohydrate form could cause greater disturbance in blend particle arrangement and densification than the dihydrate form under similar process conditions.

Finally, compression results showed that blends lubricated with MgSt-M required higher total-compression forces, ejection force, and knock-off force than those with MgSt-D. Similarity comparison based on the f₂ factor, as conducted on finished products, indicates that the blends lubricated with MgSt-D compared well with those containing MgSt-M.

### Example 9

### Superior Functional Properties of Acetaminophen Tablets Lubricated With MgSt-D

To further characterize the functional properties tablets lubricated with either MgSt-D or MgSt-M, tablets containing a high dose of acetaminophen were prepared and characterized. For this, a spray dried powder blend containing 90% acetaminophen and other excipients (available as COMPAPTM™ L from Covidien/Mallinckrodt Pharmaceuticals Company) was used. MgSt-M and MgSt-D were also obtained from Covidien/Mallinckrodt Pharmaceuticals Company. Powder blends containing 94% of COMPAP™ L, 5.4% or 5.0% microcrystalline cellulose, and lubricant at either 0.6% or 1.0% (wow) were blended in an 8 qt V-blender for 10 min. Tablets of 200 mg weight were made using a beta-press (Manesty, Merseyside, Great Britain) operating at two speeds (i.e., 50 rpm or 80 rpm). The compression and ejection force profiles were obtained using procedures similar to those described above in Example 7.

The compression force analysis for tablets containing 0.6% lubricant is presented in Table 11. To make tablets of constant weight, thickness, and hardness, the MgSt-D (50 rpm) tablets exhibited 25% lower compression force than the MgSt-M (50 rpm) tablets. Although the MgSt-M (50 rpm) tablets had lower ejection force, they exhibited some capping, sticking, and laminating problems during the tablet making process. Furthermore, while the MgSt-D (80 rpm) tablets had excellent integrity, the MgSt-M (80 rpm) tablets had capping, sticking, and laminating problems during the tablet making process. The dissolution profiles were comparable for both lubricants.

TABLE 11
Compression Force Analysis of Tablets Comprising 0.06%
Lubricant - One Way ANOVA

| Tablet | N | Mean | Standard Deviation |
|---|---|---|---|
| MgSt-D - 50 rpm | 51 | 20.384 kN | 0.477 |
| MgSt-D - 80 rpm | 51 | 20.465 kN | 0.590 |
| MgSt-M - 50 rpm | 51 | 25.202 kN | 0.132 |

S = 0.4445
R² = 96.32%
R² (adj) = 96.27%

MgSt-D tablets lubricated at 1.0% were softer than MgSt-D tablets lubricated at 0.6%. MgSt-M tablets lubricated at 1.0% experienced major capping issue and, thus, the resulting compression and ejection forces were lower than that of MgSt-D tablets. At the 1% lubrication level, therefore, it was not feasible to compare the effects due to the tablet hardness and friability.

These data show that MgSt-D is a more effective lubricant for a making tablets containing high dose acetaminophen. Using MgSt-D requires less compression force than MgSt-M to make the tablets of equivalent weight.

What is claimed is:

1. A method for lubrication of a pharmaceutical composition, the pharmaceutical composition comprising at least one pharmaceutically active ingredient and at least one excipient, the method comprising combining the pharmaceutical composition with a lubricant composition comprising at least 40% by weight of magnesium stearate dihydrate.

2. The method of claim 1, wherein the lubricant composition comprises at least 90% by weight of magnesium stearate dihydrate; and less than about 5% by weight of magnesium stearate monohydrate.

3. The method of claim 1, wherein the magnesium stearate dihydrate is a polymorph comprising stearic acid and palmitic acid.

4. The method of claim 3, wherein the weight ratio of stearic acid to palmitic acid is 2:1; the average diameter of the magnesium stearate dihydrate polymorph is from about 10 to about 20 microns; the average diameter of the pharmaceutically active ingredient is from about 200 to about 300 microns, and the average diameter of the excipient is from about 200 to about 300 microns.

5. The method of claim 1, wherein the excipient is a compressible excipient.

6. The method of claim 1, wherein the excipient is selected from the group consisting of microcrystalline cellulose, cellulose derivatives, dicalcium phosphate, lactose, starch, starch derivatives, and combinations thereof; and the pharmaceutically active ingredient is selected from the group consisting of a drug, a mineral, a vitamin, an herb, and combinations thereof.

7. The method of claim 1, wherein the pharmaceutically active ingredient and excipient comprise a form of matter selected from the group consisting of a powder, a bead, a granule, and an encapsulated particulate.

8. The method of claim 1, wherein the lubricant composition comprises at least 80% by weight of magnesium stearate dihydrate and less than about 5% by weight of magnesium stearate monohydrate; the average diameter of the magnesium stearate dihydrate is from about 5 to about 30 microns, the average diameter of the pharmaceutically active ingredient is from about 50 to about 400 microns, and the average diameter of the excipient is from about 50 to about 400 microns; the pharmaceutical composition comprises from about 0.1% to about 5% by weight of magnesium stearate dihydrate, and from about 90% to about 99% by weight of the pharmaceutically active ingredient and excipient, and the pharmaceutically active ingredient and excipient comprise a compressible powder.

9. The method of claim 1, wherein the lubricant composition comprises at least 90% by weight of magnesium stearate dihydrate and less than about 1% by weight of magnesium stearate monohydrate; the average diameter of the magnesium stearate dihydrate is from about 10 to about 20 microns, the average diameter of the pharmaceutically active ingredient is from about 200 to about 300 microns, and the average diameter of the excipient is from about 200 to about 300 microns; the pharmaceutical composition comprises from about 0.5% to about 2% by weight of magnesium stearate dihydrate, and from about 90% to about 99% by weight of the pharmaceutically active ingredient and excipient, and the pharmaceutically active ingredient and excipient comprise a compressible powder.

10. The method of claim 1, wherein the pharmaceutical composition is a dosage form selected from the group consisting of a tablet, a capsule, and a granule.

11. A pharmaceutical composition comprising:
a. at least one at least one pharmaceutically active ingredient; and
b. a lubricant composition comprising at least 40% by weight of magnesium stearate dihydrate.

12. The pharmaceutical composition of claim 11, wherein the lubricant composition comprises at least 90% by weight of magnesium stearate dihydrate and less than about 1% by weight of magnesium stearate monohydrate.

13. The pharmaceutical composition of claim 11, wherein the magnesium stearate dihydrate is a polymorph comprising stearic acid and palmitic acid, and the weight ratio of stearic acid to palmitic acid is 2:1.

14. The pharmaceutical composition of claim 11, wherein the pharmaceutical composition further comprises at least one excipient.

15. The pharmaceutical composition of claim 11, wherein the lubricant composition comprises at least 90% by weight of magnesium stearate dihydrate and less than about 1% by weight of magnesium stearate monohydrate; the average diameter of the magnesium stearate dihydrate is from about 10 microns to about 20 microns, the average diameter of the pharmaceutically active ingredient is from about 200 to about 300 microns, and the average diameter of the excipient is from about 200 to about 300 microns; and the pharmaceutical composition comprises from about 0.5% to about 2% by weight of magnesium stearate dihydrate and from about 90% to about 99% by weight of the pharmaceutically active ingredient and the excipient.

16. The pharmaceutical composition of claim 11, wherein the excipient is selected from the group consisting of microcrystalline cellulose, cellulose derivatives, dicalcium phosphate, lactose, starch, starch derivatives, and combinations thereof; the pharmaceutically active ingredient is selected from the group consisting of a drug, a mineral, a vitamin, an herb, and combinations thereof; and the pharmaceutical composition is a dosage form selected from the group consisting of a tablet, a capsule, and a granule.

17. The pharmaceutical composition of claim 16, wherein the drug is selected from the group consisting of an opioid analgesic agent, a non-opioid analgesic agent, a non-steroidal anti-inflammatory agent, and combinations thereof.

18. A composition comprising:
a. a lubricant composition comprising at least 40% by weight of magnesium stearate dihydrate; and
b. at least one excipient.

19. The composition of claim 18, wherein the lubricant composition comprises at least 90% by weight of magnesium stearate dihydrate and less than about 1% by weight of magnesium stearate monohydrate; the average diameter of the magnesium stearate dihydrate is from about 10 microns to about 20 microns and the average diameter of the excipient is from about 200 to about 300 microns; and the composition comprises from about 0.5% to about 2% by weight of magnesium stearate dihydrate and from about 90% to about 99% by weight of the excipient.

20. The composition of claim 18, wherein the excipient is a compressible excipient.
